(19) United States
(12) Patent Application Publication
(10) Pub. No.: US 2022/0395708 A1
(43) Pub. Date: Dec. 15, 2022

(54) RADIATION THERAPY DEVICES AND MAGNETIC RESONANCE GUIDED RADIATION THERAPY SYSTEMS

(71) Applicant: SHANGHAI UNITED IMAGING HEALTHCARE CO., LTD., Shanghai (CN)

(72) Inventors: Peng WANG, Shanghai (CN); Shoubo HE, Shanghai (CN); Cheng NI, Shanghai (CN); Gang PAN, Shanghai (CN); Peng CHENG, Shanghai (CN)

(73) Assignee: SHANGHAI UNITED IMAGING HEALTHCARE CO., LTD., Shanghai (CN)

(21) Appl. No.: 17/820,568

(22) Filed: Aug. 18, 2022

Related U.S. Application Data
(63) Continuation of application No. PCT/CN2020/096448, filed on Jun. 17, 2020.

(51) Int. Cl. A61N 5/10; A61B 5/055

(52) U.S. Cl. CPC A61N 5/1065; A61N 5/1081; A61B 5/055; A61N 2005/1089

(57) ABSTRACT

The present disclosure provides a radiation therapy device and a magnetic resonance guided radiation therapy system. The radiation therapy device may include an electron gun and a curved beam deflection unit. The beam deflection unit may be configured to accelerate an electron beam emitted from the electron gun within a magnetic field. The magnetic resonance guided radiation therapy system may include a radiation therapy device and a magnetic resonance imaging (MRI) device.

### CROSS-REFERENCE TO RELATED APPLICATIONS

This application is a Continuation of International Application No. PCT/CN2020/096448, filed on Jun. 17, 2020, the contents of which are hereby incorporated by reference.

### TECHNICAL FIELD

The present disclosure generally relates to medical devices and in particular, to radiation therapy devices and magnetic resonance guided radiation therapy systems.

### BACKGROUND

Radiation therapy on a tumor is currently affected by difficulties in tracking the variation (e.g., motion) of the tumor in different treatment sessions. Nowadays, various imaging techniques may be applied to provide images of the tumor before or within each treatment session. For example, a magnetic resonance imaging (MRI) device may be used in combination with a radiation therapy device to provide MRI images of the tumor. The combination of the MRI device and the radiation therapy device, which forms a therapy system, may encounter difficulties in arranging components of the MRI device (e.g., a plurality of main magnetic coils, a plurality of shielding magnetic coils) and components of the radiation therapy device (e.g., an electron accelerator) in a relatively compact space without causing interferences. Therefore, it may be desirable to provide a therapy system that provides high therapeutic quality and also has a compact structure as well.

In addition, the electron accelerator may affect the performance of the radiation therapy device. An electromagnetic field of the MRI device may affect the working of one or more components (e.g., an acceleration tube) of the electron accelerator (such as the). Therefore, it is desirable to provide an electron accelerator that can work in a magnetic field.

### SUMMARY

According to an aspect of the present disclosure, a radiation therapy device is provided. The radiation therapy device may include an electron gun and a curved beam deflection unit. The beam deflection unit may be configured to accelerate an electron beam emitted from the electron gun within a magnetic field.

In some embodiments, different portions of the beam deflection unit may have different curvatures.

In some embodiments, a curvature of a first end of the beam deflection unit close to the electron gun may be greater than a curvature of a second end of the beam deflection unit away from the electron gun.

In some embodiments, the beam deflection unit may include a plurality of acceleration cavities arranged in series. Curvatures of the plurality of acceleration cavities from close to the electron gun outward may decrease sequentially.

In some embodiments, a deflection angle of an electron beam traversing one of the plurality of acceleration cavities may be from 0° to 15°.

In some embodiments, the plurality of acceleration cavities may include a first acceleration cavity, a second acceleration cavity, a third acceleration cavity, and a fourth acceleration cavity arranged in series from close to the electron gun outward.

In some embodiments, a first deflection angle of an electron beam traversing the first acceleration cavity may be from 0° to 10°. A second deflection angle of the electron beam traversing the second acceleration cavity may be from 0° to 15°. A third deflection angle of the electron beam traversing the third acceleration cavity may be from 0° to 5°. A fourth deflection angle of the electron beam traversing the fourth acceleration cavity may be from 0° to 5°.

In some embodiments, a length of the beam deflection unit may be in a length range of 200 mm to 400 mm.

In some embodiments, a deflection angle of an electron beam traversing the beam deflection unit may be from 0° to 30°.

In some embodiments, an intensity of the magnetic field may be from 0 Gs to 50 Gs.

In some embodiments, the electron gun may include a radio frequency electron gun.

In some embodiments, the radio frequency electron gun may include a hot cathode disposed in the beam deflection unit.

In some embodiments, the hot cathode may be disposed at an end of the beam deflection unit close to the electron gun.

According to another aspect of the present disclosure, a magnetic resonance guided radiation therapy system is provided. The system may include a radiation therapy device and an MRI device. The radiation therapy device may include an electron gun and a curved beam deflection unit. The beam deflection unit may be configured to accelerate an electron beam emitted from the electron gun within a magnetic field. The MRI device may include a main magnet body including a plurality of main magnetic field coils coaxially arranged along an axis. The MRI device may include a plurality of shielding coils including a first shielding coil, a second shielding coil and a shielding coil group arranged coaxially along the axis, wherein the shielding coil group is located between the first shielding coil and the second shielding coil.

In some embodiments, the shielding coil group may include a first coil group and a second coil group arranged coaxially along the axis. The first coil group or the second coil group may include a first coil and a second coil.

In some embodiments, a direction of a current within the first coil may be opposite to a direction of a current within the second coil. A radius of the first coil or the second coil may be larger than that of the plurality of main magnetic field coils. A radius of the first coil may be greater than a radius of the second coil.

According to yet another aspect of the present disclosure, a magnetic resonance guided radiation therapy system is provided. The system may include a radiation therapy device and an MRI device. The MRI device may include a plurality of main magnetic coils. The MRI device may include a plurality of shielding magnetic coils. The MRI device may further include an annular cryostat in which the plurality of main magnetic coils and the plurality of shielding magnetic coils are coaxially arranged along an axis of the annular cryostat, the plurality of shielding magnetic coils being arranged at a larger radius from the axis than the plurality of main magnetic coils, the annular cryostat including at least one outer wall and at least one inner wall coaxial around the axis, the annular cryostat including an annular recess between the at least one outer wall and the at least one inner wall, and the annular recess having an opening formed at the at least one outer wall. The radiation therapy device may include an electron gun and a curved beam deflection unit. The beam deflection unit may be configured to accelerate an electron beam emitted from the electron gun within a magnetic field, the beam deflection unit being at least partially located within the annular recess of the annular cryostat. The radiation therapy device may include a first shielding structure configured to provide magnetic shielding for the electron gun and the beam deflection unit. The radiation therapy device may include at least one second shielding structure substantially identical to the first shielding structure, wherein the first shielding structure and the at least one second shielding structure are respectively located at selected circumferential locations within the annular recess.

In some embodiments, at least one second shielding structure may be located at an opposite circumferential position of the first shielding structure with respect to the axis.

In some embodiments, the electron gun and the curved beam deflection unit may be at least partially surrounded by the first shielding structure.

In some embodiments, the at least one second shielding structure may include more than two second shielding structures, and the first shielding structure and the at least one second shielding structure may be evenly distributed within the annular recess.

### BRIEF DESCRIPTION OF THE DRAWINGS

The present disclosure is further described in terms of exemplary embodiments. These exemplary embodiments are described in detail with reference to the drawings. Like reference numerals represent like structural components or operations. These embodiments are non-limiting exemplary embodiments, in which like reference numerals represent similar structures throughout the several views of the drawings, and wherein:

FIG. 1 is a schematic diagram illustrating an exemplary radiation therapy device 100 according to some embodiments of the present disclosure;

FIG. 2A is a schematic diagram illustrating an exemplary radiation therapy device 100 with edge coupling cavities according to some embodiments of the present disclosure;

FIG. 2B is a schematic diagram illustrating an exemplary radiation therapy device 100 with edge coupling cavities and acceleration units according to some embodiments of the present disclosure;

FIG. 3A illustrates an exemplary radiation therapy system 300 according to some embodiments of the present disclosure;

FIG. 3B illustrates another exemplary radiation therapy system 300 according to some embodiments of the present disclosure;

FIG. 4 shows an upper portion of a cross-sectional view of an exemplary therapy system 400 viewed along the Z direction according to some embodiments of the present disclosure;

FIG. 5 shows an upper portion of a cross-sectional view of another exemplary therapy system 500 viewed along the Z direction according to some embodiments of the present disclosure;

FIG. 6 shows a perspective view of an exemplary therapy system 600 according to some embodiments of the present disclosure;

FIG. 7 shows the cross-sectional view of the therapy system 700 viewed along the axial direction (i.e., the Z direction) of the cryostat according to some embodiments of the present disclosure.

### DETAILED DESCRIPTION

The following description is presented to enable any person skilled in the art to make and use the present disclosure, and is provided in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but is to be accorded the widest scope consistent with the claims.

The terminology used herein is for the purpose of describing particular exemplary embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of the present disclosure. It is to be expressly understood, however, that the drawings are for the purpose of illustration and description only and are not intended to limit the scope of the present disclosure. It is understood that the drawings are not to scale.

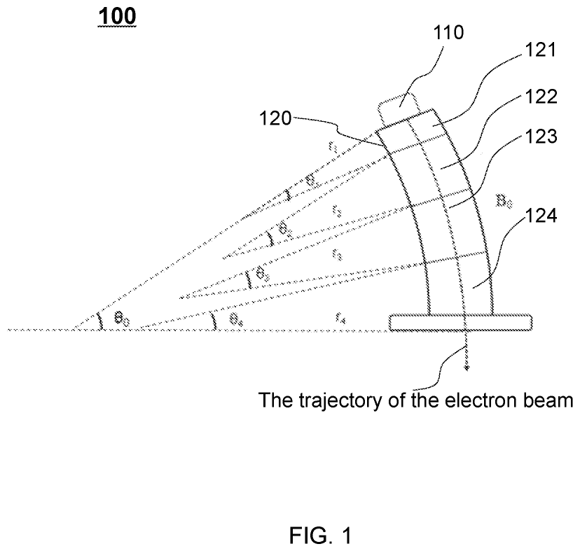

FIG. 1 is a schematic diagram illustrating an exemplary radiation therapy device 100 according to some embodiments of the present disclosure. As shown in FIG. 1, the radiation therapy device 100 may include an electron gun 110 and a curved beam deflection unit 120. A first end of the beam deflection unit 120 may be connected to the electron gun 110 to accelerate an electron beam emitted from the electron gun 110. In some embodiments, the beam deflection unit 120 may be within a magnetic field B₀. In some embodiments, a direction of the magnetic field B₀ may be perpendicular (or substantially perpendicular) to a plane where a center line of the beam deflection unit 120 is located. In some embodiments, the magnetic field B₀ may be configured to deflect the electron beam which may be accelerated by the beam deflection unit 120. The accelerated electron beam may hit a target (not shown in FIG. 1) to generate radiation rays which may be used for radiation therapy. The target may be made of aluminum, copper, stainless steel, titanium, nickel targets, or the like, or any combination thereof.

The beam deflection unit 120 may accelerate the electron beam, and a speed of the electron beam may be different in different portions of the beam deflection unit 120. Within the magnetic field B₀, the greater the speed of the electron beam at a portion of the beam deflection unit 120 is, the larger a curvature radius of a trajectory of the electron beam at that portion of the beam deflection unit 120 is, and accordingly the smaller a curvature of the trajectory of the electron beam at that portion is. In some embodiments, the trajectory of the electron beam in the beam deflection unit 120 may be determined within the magnetic field B₀ according to a calculation or simulation technology. A beam deflection unit having different curvatures in different portions of the beam deflection unit may be designed such that the electron beam may move along a predetermined trajectory in the beam deflection unit 120, thereby reducing the energy loss caused by the electron beam hitting an inner wall of the beam deflection unit 120. For example, the center line of the beam deflection unit 120 may be parallel or coincident with the predetermined trajectory of the electron beam. As another example, a space for accelerating the electron beam in the beam deflection unit 120 may include the predetermined trajectory of the electron beam.

In some embodiments, a curvature of a first end of the beam deflection unit 120 close to the electron gun 110 may be greater than a curvature of a second end of the beam deflection unit 120 away from the electron gun. A speed of the electron beam at the first end of the beam deflection unit 120 close to the electron gun 110 may be lower than a speed of the electron beam at the second end of the beam deflection unit 120 away from the electron gun so that a curvature radius of the trajectory of the electron beam at the first end of the beam deflection unit 120 close to the electron gun 110 may be less than a curvature radius of the trajectory of the electron beam at the second end of the beam deflection unit 120 away from the electron gun. Accordingly, a curvature of the beam deflection unit 120 close to the electron gun 110 may be greater than a curvature of the beam deflection unit 120 away from the electron gun 110. Therefore, the beam deflection unit 120 may match the trajectory of the electron beam.

In some embodiments, the beam deflection unit 120 may include one or more acceleration cavities arranged in series. An acceleration electric field may be present in each of the one or more acceleration cavities so that the electron beam can be accelerated therein. A speed of the electron beam in the one or more of acceleration cavities from close to the electron gun 110 outward (e.g., a direction of a motion of the electron beam) may increase sequentially. In some embodiments, the one or more acceleration cavities may be designed according to the trajectory of the electron beam. For instance, curvatures of the one or more acceleration cavities from close to the electron gun outward may be designed to decrease sequentially. A intensity of an electron field in each of the acceleration cavities may be the same or different, and the electron beam may be accelerated at the same rate or different rates in each of the acceleration cavities. In some embodiments, different portions of an acceleration cavity may have a same curvature, the acceleration cavity may be easily produced, and a space in the acceleration cavity for a movement of the electron beam may enclose and/or conform to the trajectory of the electron beam. In some embodiments, the curvatures of different portions of each of the one or more acceleration cavities may be different. For example, the curvatures of different portions of each of the one or more acceleration cavities may be the same as or similar to a trajectory of the electron beam therein. In some embodiments, a deflection angle of the electron beam traversing one of the one or more acceleration cavities may be from 0° to 15° (e.g., 1°, 3°, 5°, 10°, etc.). A deflection angle of an electron beam traversing an acceleration cavity refers to an angle between a direction along which the electron beam enters the acceleration cavity and a direction along which the electron beam exits the acceleration cavity. In some embodiments, types of different acceleration cavities of the beam deflection unit 120 may be the same or different. Exemplary types of an acceleration cavity may include an anode cavity, a beam focusing cavity, a coupling waveguide cavity, or a light velocity cavity.

Merely by way of example, the one or more acceleration cavities may include a first acceleration cavity 121, a second acceleration cavity 122, a third acceleration cavity 123, and a fourth acceleration cavity 124, which may be arranged in series from close to the electron gun 110 outward. Curvatures of the first acceleration cavity 121, the second acceleration cavity 122, the third acceleration cavity 123, and the fourth acceleration cavity 124 from close to the electron gun outward may decrease sequentially. The first acceleration cavity 121 may be connected to the electron gun 110 and configured to receive the electron beam from the electron gun 110. The electron beam may exit from the fourth acceleration cavity 124 to hit the target and generate rays for therapy radiation. In some embodiments, types of the first acceleration cavity 121, the second acceleration cavity 122, the third acceleration cavity 123, and the fourth acceleration cavity 124 may be the same or different. For example, the first acceleration cavity 121 may include an anode cavity, the second acceleration cavity 122 may include a beam focusing cavity, the third acceleration cavity 123 may include a coupling waveguide cavity, or the fourth acceleration cavity 124 may include a light velocity cavity.

In some embodiments, an electron beam traversing each of the one or more acceleration cavities may be deflected within the magnetic field B₀. In some embodiments, a first deflection angle θ₁ of the electron beam traversing the first acceleration cavity 121 may be from 0° to 10°. A second deflection angle θ₂ of the electron beam traversing the second acceleration cavity may be from 0° to 15°. A third deflection angle θ₃ of the electron beam traversing the third acceleration cavity may be from 0° to 5°. A fourth deflection angle θ4 of the electron beam traversing the fourth acceleration cavity may be from 0° to 5°. A deflection angle θ₀ of the electron beam traversing the beam deflection unit 120 including the first acceleration cavity 121, the second acceleration cavity 122, the third acceleration cavity 123, and the fourth acceleration cavity 124 may be a sum of the first deflection angle θ₁, the second deflection angle θ₂, the third deflection angle θ₃, and the fourth deflection angle θ₄. In some embodiments, the deflection angle θ₀ may be from 0° to 30°. Merely by way of example, the first deflection angle θ₁ may be 5°, the second deflection angle θ₂ may be 10°, the third deflection angle θ₃ may be 2.5°, the fourth deflection angle θ₄ may be 2.5°, and the deflection angle θ₀ may be 20°.

In some embodiments, a length of the deflection unit may be in a length range of 200 millimiters (mm) to 400 mm. For example, the length of the deflection unit may be 200 mm, 250 mm, 280 mm, 350 mm, 400 mm, etc. In some embodiments, a length, the deflection angle θ₀, and/or curvatures of different portions of the beam deflection unit 120 may be determined based on the intensity of the magnetic field B₀.

In some embodiments, the beam deflection unit 120 may include one acceleration cavity. Different portions of the acceleration cavity may have different curvatures, and the curvatures of different portions of the acceleration cavity from close to the electron gun outward may decrease sequentially to match a trajectory of an electron beam which may traverse in the acceleration cavity. In some embodiments, a deflection angle of the electron beam traversing the acceleration cavity may be from, e.g., 0° to 30°.

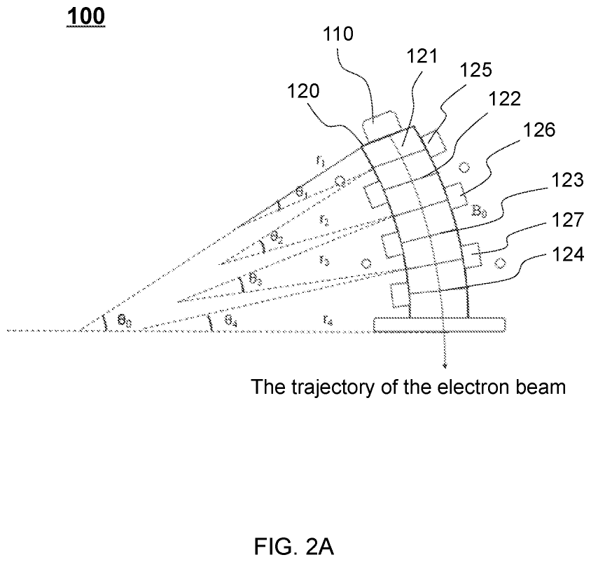

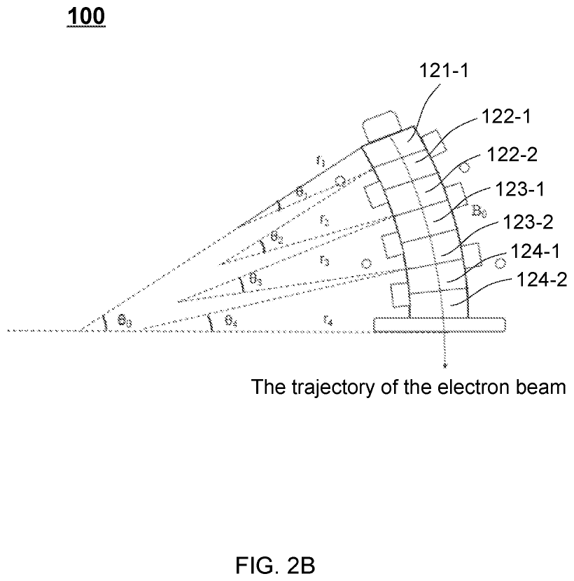

In some embodiments, the beam deflection unit 120 may include a plurality of acceleration cavities and side coupling cavities. A side coupling cavity may be connected to two adjacent acceleration cavities. A side coupling cavity may be configured to control a direction of an electric field in the side coupling cavity, thereby controlling the acceleration, deceleration, or a uniform motion of the electron beam in at least one or both of the acceleration cavities connected to the side coupling cavity. As shown in FIG. 2A, the beam deflection unit 120 may include the first acceleration cavity 121, the second acceleration cavity 122, the third acceleration cavity 123, the fourth acceleration cavity 124, a first side coupling cavity 125 connecting the first acceleration cavity 121 and the second acceleration cavity 122, a second side coupling cavity 126 connecting the second acceleration cavity 122 and the third acceleration cavity 123, and a third side coupling cavity 127 connecting the third acceleration cavity 123 and the fourth acceleration cavity 124. In some embodiments, the beam deflection unit 120 may include a standing wave acceleration tube. Each of the acceleration cavities of the beam deflection unit 120 may include one or more acceleration units. As shown in FIG. 2B, the first acceleration cavity 121 may include one acceleration unit 121-1, and the second acceleration cavity 122 may include two acceleration unit 122-1, 122-2, the third acceleration cavity 123 may include two acceleration unit 123-1, 123-2, and the fourth acceleration cavity 124 may include two acceleration units 124-1, 124-2, respectively. When an acceleration cavity includes two or more acceleration units, a side coupling cavity may be disposed between every two adjacent acceleration units.

In some embodiments, an intensity of the magnetic field B₀ may be from 0 Gs to 50 Gs. The magnetic field B₀ may be generated by an MRI device. In some embodiments, the magnetic field B₀ may include a uniform magnetic field, a non-uniform field, or a partially non-uniform field that is uniform in at least one portion of the magnetic field B₀ and non-uniform in other portions of the magnetic field B₀.

In some embodiments, the electron gun may include a radio frequency electron gun. The radio frequency electron gun may include a heating component (e.g., a heating filament) and a hot cathode (not shown in FIG. 2A). The heating component may be configured to heat the hot cathode to generate the electron beam. In some embodiments, the hot cathode may be disposed in the beam deflection unit 120. For example, the hot cathode may be disposed on an acceleration cavity (e.g., the first acceleration cavity 121) close to the electron gun. When the hot cathode is heated by the heating component to a temperature at which the hot cathode may emit electrons, the electrons on a surface of the hot cathode may be accelerated by a radio frequency electromagnetic field of the first acceleration cavity 121. In this case, problems such as decreasing an emissivity and an emission density caused by injecting the electron gun into the acceleration cavity, or the like, may be solved. Using the radio frequency electron gun may improve the efficiency of the radiation therapy device within the magnetic field B₀. Due to an effect of the magnetic field B₀, electrons of the electron beam that are reversely accelerated may travel at the opposite direction than when the electrons are emitted from electron gun, thereby avoiding that the reversely accelerated electrons impinge on the surface of the electron gun and improving the stability of the radio frequency electron gun. In some embodiments, the electron gun 110 may include a grid-controlled electron gun. An anode of the grid-controlled electron gun may be connected to or disposed in the acceleration cavity that is at the first end of the beam deflection unit 120 close to the electron gun 110. In some embodiments, the electron gun 110 may include other types of electron guns (e.g., a Kano electron gun, etc.), and are not limited to those exemplified herein.

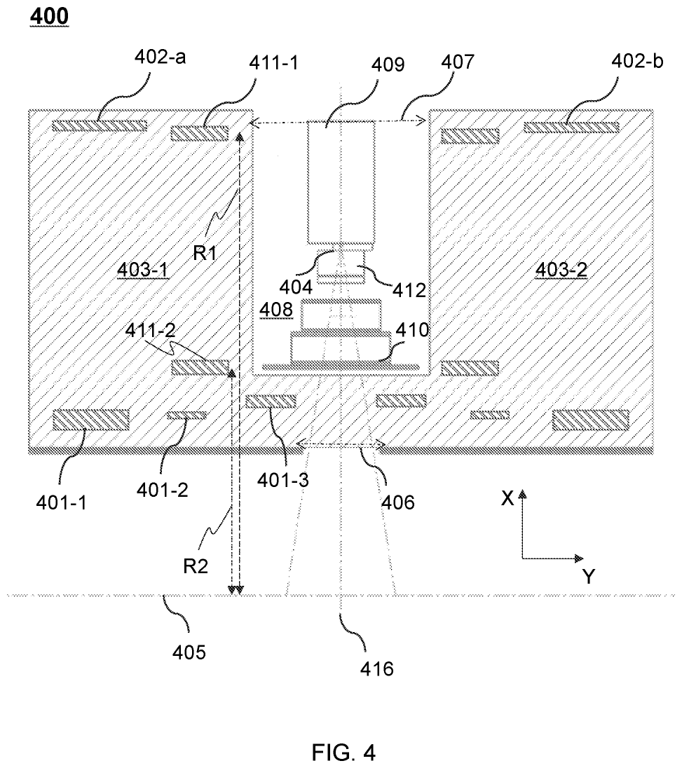

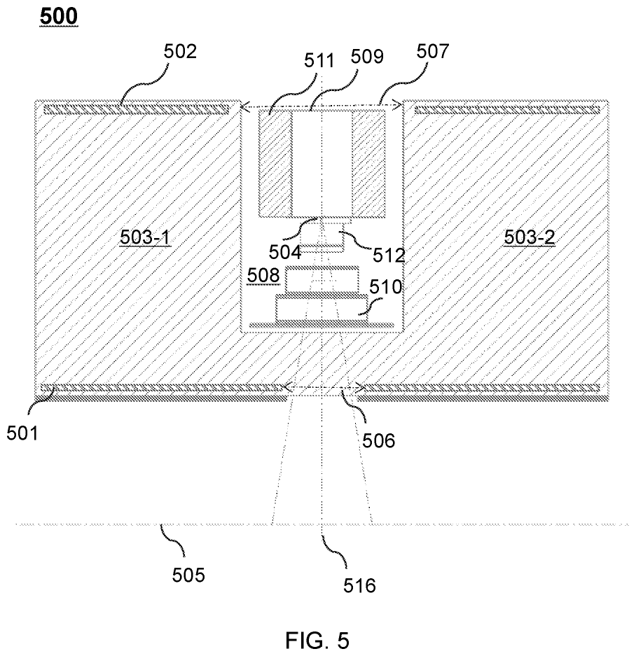

Embodiments of the present disclosure relate to the beam deflection unit 120 and/or the electron gun 110, which may be operated within a magnetic field, thereby effectively reducing the effect of the magnetic field of the MRI device to the radiation therapy device. To further reduce the effect of the magnetic field of the MRI device to the radiation therapy device, the present disclosure provides an active shielding structure (as shown in FIG. 4) which may be configured to optimize a magnetic body in the MRI device to weaken a magnetic field generated by the MRI device and located around the radiation therapy device. The present disclosure also provides a passive shielding structure (e.g., as shown in FIGS. 5-7). Embodiments of the passive shielding structure may be disposed around the radiation therapy device, thereby weakening the magnetic field generated by the MRI device and located around the radiation therapy device. In some embodiments, the radiation therapy system may include the active shielding structure, the passive shielding structure, or the like, or any combination thereof.

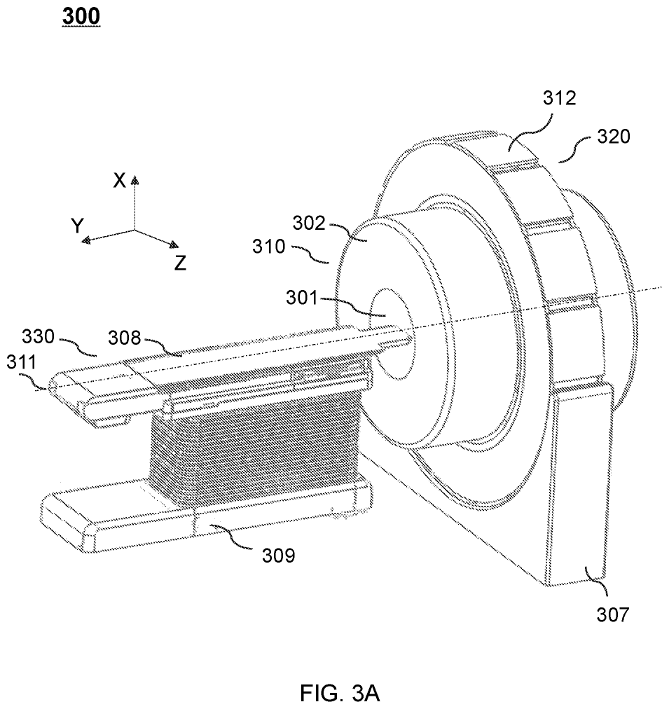

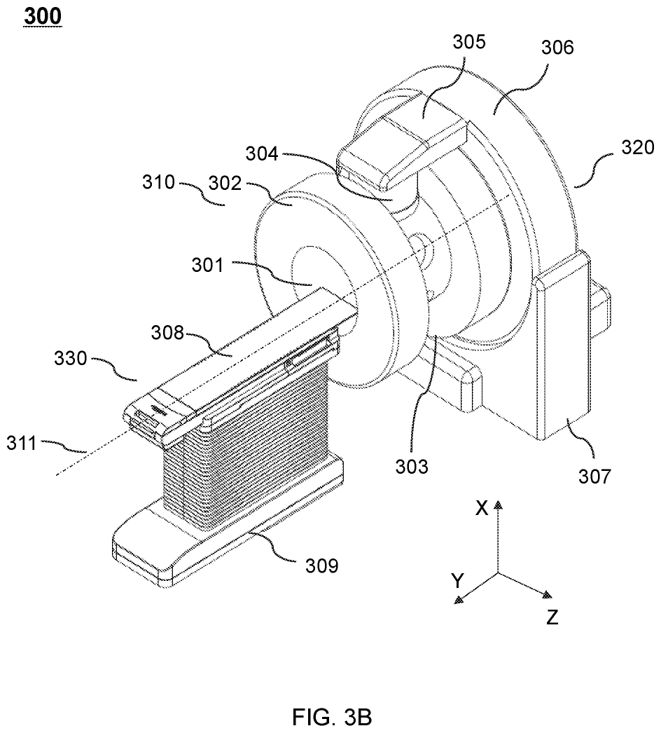

FIG. 3A illustrates an exemplary radiation therapy system 300 according to some embodiments of the present disclosure. As illustrated in FIG. 3A, the radiation therapy system 300 may include an MRI device 310, a radiation therapy device 320, and a treatment table 330.

The MRI device 310 may include a bore 301, a main magnetic body 302, one or more gradient coils (not shown), and one or more radiofrequency (RF) coils (not shown). The MRI device 310 may be configured to acquire image data from an imaging region. For example, the image data may relate to the treatment region associated with a lesion, e.g., a tumor. In some embodiments, the MRI device 310 may be a permanent magnet MRI scanner, a superconducting electromagnet MRI scanner, or a resistive electromagnet MRI scanner, etc., according to the types of the main magnetic body 302. In some embodiments, the MRI device 310 may be a high-field MRI scanner, a mid-field MRI scanner, and a low-field MRI scanner, etc., according to the intensity of the magnetic field. In some embodiments, the MRI device 310 may be of a closed-bore (cylindrical) type, an open-bore type, or the like.

The main magnetic body 302 may have the shape of an annulus and may generate a static magnetic field B1. The main magnetic body 302 may be of various types including, for example, a permanent magnet, a superconducting electromagnet, a resistive electromagnet, etc. The superconducting electromagnet may include niobium, vanadium, technetium alloy, etc.

The one or more gradient coils may generate magnetic field gradients to the main magnetic field B1 in the X, Y, and/or Z directions (or axes). In some embodiments, the one or more gradient coils may include an X-direction (or axis) coil, a Y-direction (or axis) coil, a Z-direction (or axis) coil, etc. For example, the Y-direction coil may be designed based on a circular (Maxwell) coil, the Z-direction coil and the X-direction coil may be designed on the basis of the saddle (Golay) coil configuration. As used herein, the Z direction may also be referred to as the readout (RO) direction (or a frequency encoding direction), the X direction may also be referred to as the phase encoding (PE) direction, the Y direction may also be referred to as the slice-selection encoding direction. In the present disclosure, the readout direction and the frequency encoding direction may be used interchangeably.

Merely by way of example, the gradient magnetic fields may include a slice-selection gradient field corresponding to the Y-direction, a phase encoding (PE) gradient field corresponding to the X-direction, a readout (RO) gradient field corresponding to the Z-direction, etc. The gradient magnetic fields in different directions may be used to encode the spatial information of MR signals. In some embodiments, the gradient magnetic fields may also be used to perform at least one function of flow encoding, flow compensation, flow dephasing, or the like, or any combination thereof.

The one or more RF coils may emit RF pulses to and/or receive MR signals from a subject (e.g., a body, a substance, an object) being examined. As used herein, an RF pulse may include an excitation RF pulse and a refocusing RF pulse. In some embodiments, the excitation RF pulse (e.g., a 90-degree RF pulse) may tip a magnetization vector away from the direction of the main magnetic field B1. In some embodiments, the refocusing pulse (e.g., a 180-degree RF pulse) may rotate dispersing spins isochromaticly about an axis in the transverse plane so that magnetization vector may rephase at a later time. In some embodiments, the RF coil may include an RF transmitting coil and an RF receiving coil. The RF transmitting coil may emit RF pulse signals that may excite the nucleus in the subject to resonate at the Larmor frequency. The RF receiving coil may receive MR signals emitted from the subject. In some embodiments, the RF transmitting coil and RF receiving coil may be integrated into one single coil, for example, a transmitting/receiving coil. The RF coil may be one of various types including, for example, a quotient difference (QD) orthogonal coil, a phase-array coil, etc. In some embodiments, different RF coils may be used for the scanning of different parts of a body being examined, for example, a head coil, a knee joint coil, a cervical vertebra coil, a thoracic vertebra coil, a temporomandibular joint (TMJ) coil, etc. In some embodiments, according to its function and/or size, the RF coil may be classified as a volume coil and a local coil. For example, the volume coil may include a birdcage coil, a transverse electromagnetic coil, a surface coil, etc. As another example, the local coil may include a solenoid coil, a saddle coil, a flexible coil, etc.

The radiation therapy device 320 may include a drum 312 and a base 307. The drum 312 may have the shape of an annulus. The drum 312 may be disposed around the main magnetic body 302 and intersect the main magnetic body 302 at a central region of the main magnetic body 302 along the axis 311 of the bore 301. The drum 312 may accommodate and support a radiation source that is configured to emit a radiation beam towards the treatment region in the bore 301. The radiation beam may be an X-ray beam, an electron beam, a proton ray source, etc. The drum 312, together with the radiation source mounted thereon, may be able to rotate around the axis 311 of the bore 301 and/or a point called the isocenter. Merely by way of example, the drum 312, together with the radiation source mounted thereon, may be able to rotate any angle, e.g., 90 degrees, 180 degrees, 360 degrees, 450 degrees, 540 degrees, around the axis 311. The drum 312 may be further supported by the base 307.

It should be noted that the above description is merely provided for the purposes of illustration, and not intended to limit the scope of the present disclosure. For persons having ordinary skills in the art, multiple variations or modification may be made under the teaching of the present disclosure. For example, the radiation therapy device 320 may further include a electron gun configured to emit electrons, a curved beam deflection unit configured to accelerate electrons, ions, or protons, a dose detecting device, a temperature controlling device (e.g., a cooling device), a multiple layer collimator, or the like, or any combination thereof. However, those variations and modifications do not depart from the scope of the present disclosure.

The treatment table 330 may include a platform 308 and a base frame 309. In some embodiments, the platform 308 may move along the horizontal direction and enter into the bore 301 of the MRI device 310. In some embodiments, the platform 308 may move two-dimensionally, three-dimensionally, four-dimensionally, five-dimensionally or six-dimensionally. In some embodiments, the platform 308 may move according to the variance (e.g., position change) of the tumor estimated by, for example, a real-time MRI image obtained during a treatment.

In some embodiments, a subject may be placed on the platform 308 and sent into the MRI device 310. In some embodiments, the subject may be a human patient. The human patient may lie on the back, lie in prone, lie on the side, etc., on the platform 308.

During the treatment, the drum 312 may be set to rotate around the main magnetic body 302. In some embodiments, the main magnetic body 302 may include a recess (not shown) at its outer wall. The recess may be disposed around the entire circumference of the main magnetic body 302. For example, the recess may have the shape of an annulus surrounding the main magnetic body 302, thus accommodating at least part of the drum 312. In some embodiments, the recess may be disposed around part of the circumference of the main magnetic body 302. For example, the recess may have the shape of one or more arcs around the main magnetic body 302.

In some embodiments, at least a portion of the radiation source is within the recess. This arrangement may reduce the distance between the radiation source and the axis 311 of the bore 301 along the radial direction of the main magnetic body 302. In some embodiments, the radiation source may move along an entire path of rotation within the recess. In some embodiments, the radiation source may move along a path of rotation within the recess that is a portion of, but not the entire, circle, such as a semicircle or ¾ circle or ⅘ circle. Under such situations, the radiation source may move clockwise and then anti-clockwise during treatment, and the table may also move. The radiation source may generate the radiation beam according to one or more parameters. Exemplary parameters may include a parameter of the radiation beam, a parameter of the radiation source, or a parameter of the platform 308. For example, the parameter of the radiation beam may include an irradiating intensity, an irradiating angle, an irradiating distance, an irradiating area, an irradiating time, an intensity distribution, or the like, or any combination thereof. The parameter of the radiation source may include a position, a rotating angle, a rotating speed, a rotating direction, the configuration of the radiation source, or the like, or any combination thereof. In some embodiments, the generation of the radiation beam by the radiation source may take into consideration energy loss of the radiation beam due to, e.g., the main magnetic body 302 located in the pathway of the radiation beam that may absorb at least a portion of the radiation beam. For example, the irradiating intensity of the radiation beam may be set larger than that in the situation in which there is no energy loss due to, e.g., the absorption by the main magnetic body 302 so as to compensate the energy loss such that the radiation beam of a specific intensity may impinge on a lesion or treatment region (e.g., a tumor).

FIG. 3B illustrates another exemplary radiation therapy system 300 according to some embodiments of the present disclosure. Compared with the therapy system 300 described in FIG. 3A, the therapy system 300 may use a gantry 306 instead of the drum 312. The gantry 306 may be disposed at one side of the main magnetic body 302. A treatment head 304 may be installed on the gantry 306 via a treatment arm 305. The treatment head 304 may accommodate the radiation source. The gantry 306 may be configured to rotate the treatment head 304 around the axis 311 of the bore 301.

As shown in FIG. 3B, a recess 303 may be formed on the outer wall of the main magnetic body 302 and have the shape of an annulus. The recess 303 may accommodate at least a portion of the treatment head 304 and provide a path for the treatment head 304 to rotate. This arrangement may reduce the distance between the treatment head 304 and the axis 311 of the bore 301 along the radial direction of the main magnetic body 302. In some embodiments, the reduction of the distance between the treatment head 304 and the axis 311 of the bore 301 may cause an increase of the radiation dose that may reach the lesion or treatment region (e.g., a tumor), leading to an enhancement in the therapeutic efficiency. In some embodiments, the width of the recess 303 along the Y direction (i.e., the axial direction of the main magnetic body 302) may be no less than the width of the treatment head 304 along the Y direction.

It should be noted that the above description of the therapy system 300 is merely provided for the purposes of illustration, and not intended to limit the scope of the present disclosure. For persons having ordinary skills in the art, multiple variations and modifications may be made under the teachings of the present disclosure. For example, the assembly and/or function of the therapy system 300 may vary or change according to a specific implementation scenario. In some embodiments, the main magnetic body 302 of the MRI device 310 may also rotate relative to the treatment head 304. For example, the radiation therapy device 320 and the MRI device 310 may synchronously or asynchronously rotate around a same axis (e.g., the axis 311). However, those variations and modifications do not depart from the scope of the present disclosure.

FIG. 4 shows an upper portion of a cross-sectional view of an exemplary therapy system 400 viewed along the Z direction according to some embodiments of the present disclosure. The therapy system 400 may include an MRI device that is configured to generate MRI data and a radiation therapy device that is configured to apply therapeutic radiation.

As shown in FIG. 4, the MRI device may include a plurality of main magnetic field coils 401 (e.g., first main magnetic field coils 401-1, second main magnetic field coils 401-2, third main magnetic field coils 401-3), a plurality of shielding coils (e.g., shielding coils 402, shielding coils 411-1, shielding coils 411-2), and a cryostat 403. The shielding coils 402 may include a first pair of shielding coils of a first size, i.e., a first shielding coil 402a and a second shielding coil 402-b. The shielding coils 411-1 may include a second pair of shielding coils of a second size. The shielding coils 411-2 may include a third pair of shielding coils of a third size. The first size, the second size and the third size may be different from each other. The shielding coils 411-1 (i.e., the second pair of shielding coils) may be close to the shielding coils 402 (i.e., the first pair of shielding coils). In some embodiments, the shielding coils 411-1 (also referred to as first coils) and the shielding coils 411-2 (also referred to as second coils) may also be referred to as a shielding coil group 411.

The plurality of main magnetic field coils 401, the shielding coils 402, and the shielding coil group 411 may be accommodated in the cryostat 403 and maintained in the superconductive state under a certain condition (e.g., when all the coils are merged in a cooling medium in the cryostat 403).

The cryostat 403 may have the shape of an annulus with an axis 405 (e.g., the axis 311 in FIG. 3A). The plurality of main magnetic field coils 401 may be arranged coaxially along the axis 405 to generate a uniform magnetic field (e.g., a static magnetic field B1) within a specific region (e.g., the region within the bore 301) when the plurality of main magnetic field coils 401 carry an electric current along a first direction. In some embodiments, the first main magnetic field coils 401-1, the second main magnetic field coils 401-2, and the third main magnetic field coils 401-3 may have the same radius or different radiuses.

The shielding coils 402 may also be arranged coaxially along the axis 405 at a larger radius from the axis 405 than the plurality of main magnetic field coils 401. That is, a radius of each of the first shielding coil 402-a and the second shielding coil 402-b may be larger than that of each of the plurality of main magnetic field coils 401. The shielding coils 402 may carry an electric current along a second direction that is opposed to the first direction. The shielding coils 402 (i.e., the first pair of shielding coils) may help shield the magnetic field generated by the plurality of main magnetic field coils 401 on a region outside the MRI device.

The shielding coil group 411 may also be arranged coaxially along the axis 405 at a larger radius from the axis 405 than the plurality of main magnetic field coils 401. That is, a radius of each of the first coils 411-1 and second coils 411-2 may be larger than that of each of the plurality of main magnetic field coils 401. A direction of a current within each of the first coils 411-1 may be opposite to a direction of a current within each of the second coils 411-2. For example, each of the first coils 411-1 may include a radius designated as R1, and each of the second coils 411-2 may include a radius designated as R2, wherein R1 is greater than R2. Each of the first coils 411-1 may carry an electric current along the first direction, and each of the second coils 411-2 may carry an electric current along the second direction. That is, the direction of the electric current within the first coils 411-1 (i.e., the second pair of shielding coils) may be the same as that of the plurality of main magnetic field coils 401, and the direction of the electric current within the second coils 411-2 (i.e., the third pair of shielding coils) may be opposite to that of the plurality of main magnetic field coils 401 (i.e., the direction of a current within the third pair of shielding coils being opposite to the direction of a current within the second pair of shielding coils). In some embodiments, a shielding coil of the second pair of shielding coils (i.e., a first coil 411-1) may be concentric with a shielding coil of the third pair of shielding coils (i.e., a second coil 411-2). The first coil 411-1 and the second coil 411-2 that are arranged concentrically may also be referred to as a coil group of the shielding coil group 411. As shown in FIG. 4, the shielding coil group 411 may include a first coil group and a second coil group.

In some embodiments, the shielding coil group 411 may be configured to shield the magnetic field produced by the MRI device (e.g., the main magnetic field coils, the magnetic shielding coils, the gradient coils) in cases that one or more components of the radiation therapy device (e.g., a curved beam deflection unit, an electron gun, a multi-leaf collimator) may be affected by the magnetic field produced by the MRI device on an annular region. The annular region may have the shape of an annulus with the axis 405. The annular region may include a virtual outer wall with a radius of R1 and a virtual inner wall with a radius of R2. That is, the depth of the annular region (i.e., the thickness of the annular region in the radial direction) which is defined as the distance from the virtual outer wall to the virtual inner wall in the radial direction may be equal to R1 minus R2 (R1−R2). For example, the shielding coil group 411 (e.g., the second pair of shielding coils 411-1, or the third pair of shielding coils 411-2) may be configured to shield a magnetic field between the shielding coils 402 (i.e., the first pair of shielding coils) and the main magnetic field coils 401. As another example, the shielding coil group 411 (e.g., the second pair of shielding coils 411-1, the third pair of shielding coils 411-2) may be configured to reduce a magnetic field on a region within a recess (e.g., a recess 408) of the annular cryostat 403.

In some embodiments, a magnitude of the electric current in each coil of the shielding coil group 411 may be the same, i.e., each of the first coils 411-1 may have the electric current of a same magnitude as each of the second coils 411-2. Taking the first direction that is perpendicular to the X-Y plane pointing inwards as an example, the second direction may be perpendicular to the X-Y plane (in which the X and Y directions are illustrated in FIGS. 3A and 3B) pointing outward. For the annular region, the magnetic field produced by the plurality of main magnetic field coils 401 (also referred to as a first magnetic field) in the annular region may be along the Y direction (as illustrated in FIGS. 3A and 3B), and the magnetic field produced by the shielding coil group 411 (also referred to as the second magnetic field) in the annular region may be opposite to the Y direction. The magnitude of the first magnetic field may be configured to equal to or approximately equal to the second magnetic field by adjusting the magnitude of the electric current in each coil of the shielding coil group 411 to a proper magnitude. By setting the electric current of proper magnitude in each coil of the shielding coil group 411, the first magnetic field and the second magnetic field may cancel out each other such that the magnetic field in the annular region may be equal to or less than a threshold field (e.g., a zero net field). The threshold field may be set by an operator or according to a default setting of the radiation therapy system 400, and may be adjustable in different situations. For a region of the main magnetic field B1 that produced by the plurality of main magnetic field coils 401, the magnetic field produced by the shielding coil group 411 (also referred to as the third magnetic field) in the region of the main magnetic field B1 may be a magnetic field equal to or less than the threshold field, as the first coils 411-1 and the second coils 411-2 may produce two magnetic fields of approximately the same magnitude and in opposite directions in the region of the main magnetic field B1 so that the two magnetic fields may substantially cancel out each other. Thus, the main magnetic field B1 are not affected by the protection measures achieved by the first coils 411-1 and the second coils 411-2 producing two magnetic fields having the same magnitude and in opposite direction.

As shown in FIG. 4, the cryostat 403 may include two chambers (e.g., the left chamber 403-1 and the right chamber 403-2 for brevity). The two chambers may be located on opposite sides of the cryostat 403 along the axial direction (i.e., the direction of the axis 405) and may be operably connected by a neck portion between the two chambers to establish fluid communication between the two chambers. The neck portion may have a smaller radial size than the two chambers. Each chamber may have the shape of an annulus with a different outer wall. In some embodiments, the outer wall of a chamber may refer to the outermost surface of the chamber that has the shape of a ring. The two chambers and the neck portion may share a same inner wall, i.e., the inner wall of the cryostat 403. In some embodiments, the inner wall of a chamber may refer to the innermost surface of the chamber that has the shape of a ring. In some embodiments, each chamber may accommodate at least one of the plurality of main magnetic field coils 401, at least one of the shielding coils 402, and at least one of the first coils 411-1 and the second coils 411-2 of the shielding coil group 411. For example, at least one of the plurality of main magnetic field coils 401 may be arranged near the inner wall of the left chamber 403-1, at least one of the shielding coils 402 (e.g., the first shielding coil 402-a) may be arranged near the outer wall of the left chamber 403-1 as illustrated in FIG. 4, at least one of the first coils 411-1 and the second coils 411-2 of the shielding coil group 411 (e.g., the first coil group) may be arranged near the outer wall of the left chamber 403-1 and close to the neck portion. A gap 406 may be formed between the main magnetic field coils arranged in the left chamber 403-1 and the main magnetic field coils arranged in the right chamber 403-2 as illustrated in FIG. 4, allowing the radiation beam produced by the radiation therapy device to pass through. The two chambers may be in fluid communication with each other through the neck portion between them. The cryostat 403 may contain a cooling medium in which the plurality of main magnetic field coils 401 and the shielding coils 402 are merged to achieve the superconducting state. In some embodiments, the magnetic fields coils and the shielding coils may be replaced by permanent magnets. The direction of the magnetic field produced by the permanent magnets that replace the magnetic fields coils may be opposite to the direction of the magnetic field by the permanent magnets that replace the shielding coils.

The cryostat 403 may have a recess 408 at a radial position between the inner wall of the cryostat 403 and the outer walls of the different chambers of the cryostat 403. The recess 408 may have an opening 407 formed between the outer walls of the two chambers of the cryostat 403. The recess 408 may have the shape of an annulus when viewed in a perspective view. The annulus may have the same width or different widths (i.e., the size in the axial direction) at different radial positions. The recess 408 may have a depth (i.e., the thickness of the annulus in the radial direction) which is defined as the distance from the opening 407 to the outermost surface of the neck portion of the cryostat 403 in the radial direction. As show in FIG. 4, the third pair of shielding coils 411-2 may be arranged close to a bottom of the recess 408, and the second pair of shielding coils 411-1 may be arranged close to the opening of the recess 408.

The recess 408 may be configured to accommodate the components of the radiation therapy device. As shown in FIG. 4, the recess 408 may accommodate at least a portion of a radiation source, wherein the radiation source includes an electron gun (not shown), a curved beam deflection unit 409, a collimator 412, a target 404 and a multi-leaf collimator (MLC) 410.

The curved beam deflection unit 409 may be configured to accelerate charged subatomic particles or ions to a high speed. In some embodiments, the curved beam deflection unit 409 may accelerate electrons using microwave technology. For example, the curved beam deflection unit 409 may accelerate electrons in an electron beam with energy levels in the range between 4 MeV to 22 MeV using high RF electromagnetic waves.

The curved beam deflection unit 409 may be mounted to a gantry or a drum (e.g., the gantry 306 or the drum 312) that is capable of rotating around the axis 405 and may enable the radiation beam to be emitted from a certain range of the circumferential positions, or an arbitrary circumferential position. As shown in FIG. 4, the gantry or the drum may rotate to a first position where the curved beam deflection unit 409 may be located above the axis 405. The curved beam deflection unit 409 may include an accelerating waveguide (tube) whose axis is perpendicular to the axis 405. The one skilled in the art could readily understand that electrons described herein could be replaced by other particles in other embodiments.

The target 404 may be configured to receive the accelerated charged subatomic particles or ions (e.g., an electron beam) to produce the radiation beam for the therapeutic radiation. For example, the electron beam may collide with the target 404 to generate high-energy X-rays according to the bremsstrahlung effect. In some embodiments, the target 404 may be located near the exit window of the curved beam deflection unit 409 to receive the accelerated electron beam. In some embodiments, the target 404 may be made of one or more materials including aluminum, copper, silver, tungsten, or the like, or an alloy thereof, or any combination thereof. For instance, the target 404 may be made of a composite material including a composite of tungsten and copper, a composite of tungsten and silver, a composite of tungsten and aluminum, or the like, or any combination thereof. The one skilled the art could readily understand that the target is not necessary for the treatment using the electron beam.

The radiation beam from the target 404 may pass through the collimator 412 to form a beam of a specific shape (e.g., cone beam). In some embodiments, the collimator 412 may include a primary collimator, a flattening filter and at least one secondary collimator.

The MLC 410 may be configured to reshape the radiation beam. For example, the MLC 410 may adjust the irradiating shape, the irradiating area, etc., of the radiation beam. The MLC 410 may be placed anywhere on the path of the radiation beam. For example, the MLC 410 may be placed close to the curved beam deflection unit 409 as shown in FIG. 4. Thus, the radiation beam, after being reshaped by the MLC 410, may further pass through the neck portion of the cryostat 403 and the gap 406 between the plurality of main magnetic field coils to arrive at the lesion or treatment region (e.g., tumor). As another example, the MLC 410 may be placed by a relatively long distance away from the curved beam deflection unit (e.g., as such that the MLC 410 may be closer to, e.g., the patient to be radiated).

The MLC 410 may stay fixed relative to the curved beam deflection unit 409, thus rotating together with the curved beam deflection unit 409 around the axis 405. The MLC 410 may include a plurality of individual leaves of high atomic numbered materials (e.g., tungsten) moving independently in and out of the path of the radiation beam in order to selectively block it. The shape of the radiation beam may vary when the plurality of individual leaves move in and out, forming different apertures that may be adapted to the cross section of the lesion or treatment region including, e.g., a tumor, viewed from an axis of the radiation beam (i.e., the vertical dotted line 416 shown in FIG. 4). In some embodiments, the MLC 410 may include one or more layers of leaves. For example, the MLC 410 may have only one layer of leaves and the height of the MLC 410 along the axis of the radiation beam from the top of the MLC 410 to the bottom of the MLC 410 may be between 7 and 10 centimeters. As another example, the MLC 410 may include two layers and the height of the MLC 410 may be at least 15 centimeters.

As shown in FIG. 4, the radiation therapy device may be located coaxially and/or radially between the first coil group and the second coil group. The radiation therapy device may rotate within the annular region to reduce the influence of components of the radiation therapy device (e.g., the electron gun (not shown), the curved beam deflection unit 409, the collimator 412, the target 404, the MLC 410) by the magnetic field produced by the MRI device. The depth of the annular region (i.e., R1−R2) may be equal to or greater than a height of a portion of the radiation therapy device (e.g., a height of at least a portion of the radiation source) which is defined as the distance from the top of the portion of the radiation therapy device to the bottom of the portion of radiation therapy device in the radial direction.

In some embodiments, the depth of the annular region may accommodate only a portion of components of the radiation therapy device to protect the portion of components from being influenced by the magnetic field produced by the MRI device as possible. For example, the annular region may accommodate the target 404, the collimator 412 and the MLC 410. The curved beam deflection unit 409 may be out of the annular region, as the accelerating waveguide (tube) of the curved beam deflection unit 409 may be surrounded by a shielding structure or the curved beam deflection unit 409 may be located in a relatively long distance away from the plurality of main magnetic field coils 401. The shielding structure may include a plurality of shielding layers to shield the magnetic field produced by the MRI device in case that the electrons may be influenced by the magnetic field and/or absorb the radiation produced by the radiation beam of the curved beam deflection unit 409 in case that the plurality of main magnetic field coils 401 is influenced. As another example, the annular region may accommodate the curved beam deflection unit 409 and the target 404. The collimator 412 and the MLC 410 may be out of the annular region.

FIG. 5 shows an upper portion of a cross-sectional view of another exemplary therapy system 500 viewed along the Z direction according to some embodiments of the present disclosure. The therapy system 500 may include a magnetic resonance imaging (MRI) device that is configured to generate MRI data and a radiation therapy device that is configured to apply therapeutic radiation.

As shown in FIG. 5, the MRI device may include a plurality of main magnetic coils 501, a plurality of shielding magnetic coils 502, and a cryostat 503.

The plurality of main magnetic coils 501 and the plurality of shielding magnetic coils 502 may be accommodated in the cryostat 503 and maintained in the superconductive state under a certain condition (e.g., when all the coils are merged in a cooling medium in the cryostat 503).

The cryostat 503 may have the shape of an annulus with an axis 505 (e.g., the axis 311 in FIG. 3A). The plurality of main magnetic coils 501 may be arranged coaxially along the axis 505 to generate a uniform magnetic field (e.g., a static magnetic field B1) within a specific region when the plurality of main magnetic coils 501 carry an electric current along a first direction.

The plurality of shielding magnetic coils 502 may also be arranged coaxially along the axis 505 at a larger radius from the axis 505 than the plurality of main magnetic coils 501. The plurality of shielding magnetic coils 502 may carry an electric current along a second direction that is opposed to the first direction. The plurality of shielding magnetic coils 502 may help shield the magnetic field generated by the plurality of main magnetic coils 501 on a region outside the MRI device.

As shown in FIG. 5, the cryostat 503 may include two chambers (e.g., the left chamber 503-1 and the right chamber 503-2 for brevity). The two chambers may be located on opposite sides of the cryostat 503 along the axial direction (i.e., the direction of the axis 505) and may be connected by a neck portion between the two chambers. The neck portion may have a smaller radial size than the two chambers. Each chamber may have the shape of an annulus with a different outer wall. In some embodiments, the outer wall may refer to the outermost surface of each chamber that has the shape of a ring. The two chambers and the neck portion may share a same inner wall, i.e., the inner wall of the cryostat 503. In some embodiments, the inner wall may refer to the innermost surface of each chamber that also has the shape of a ring. In some embodiments, each chamber may accommodate at least one of the plurality of main magnetic coils 501 and at least one of the plurality of shielding magnetic coils 502. For example, at least one of the plurality of main magnetic coils 501 may be arranged near the inner wall of the left chamber 503-1, and at least one of the plurality of shielding magnetic coils 502 may be arranged near the outer wall of the left chamber 503-1. A gap 506 may be formed between the main magnetic coils arranged in the left chamber 503-1 and the main magnetic coils arranged in the right chamber 503-2 as illustrated in FIG. 5, allowing the radiation beam produced by the radiation therapy device to pass through. The two chambers may be in fluid communication with each other through the neck portion between them. The cryostat 503 may contain cooling mediums in which the plurality of main magnetic coils 501 and the plurality of shielding magnetic coils 502 are merged to achieve the superconducting state.

The cryostat 503 may have a recess 508 at a radial position between the inner wall of the cryostat 503 and the outer walls of the different chambers of the cryostat 503. The recess 508 may have an opening 507 formed between the outer walls of the two chambers of the cryostat 503. The recess 508 may have the shape of an annulus when viewed in a perspective view. The annulus may have same or different widths (i.e., the size in the axial direction) at different radial positions. The recess 508 may have a depth (i.e., the thickness of the annulus in the radial direction) which is defined as the distance from the opening 507 to the outermost surface of the neck portion of the cryostat 503 in the radial direction.

The recess 508 may be configured to accommodate the components of the radiation therapy device As shown in FIG. 5, the recess 508 may accommodate a radiation source that includes an electron gun (not shown), a curved beam deflection unit 509, a shielding structure 511, a collimator 512, a target 504 and a multi-leaf collimator (MLC) 510.

The curved beam deflection unit 509 may be configured to accelerate charged subatomic particles or ions to a high speed. In some embodiments, the curved beam deflection unit 509 may accelerate electrons using microwave technology. For example, the curved beam deflection unit 509 may accelerate electrons in an electron beam with energy level in the range between 4 MeV to 22 MeV using high RF electromagnetic waves.

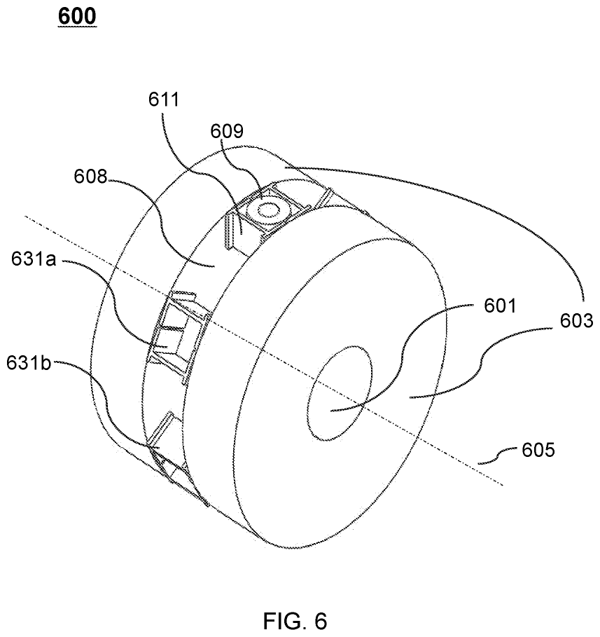

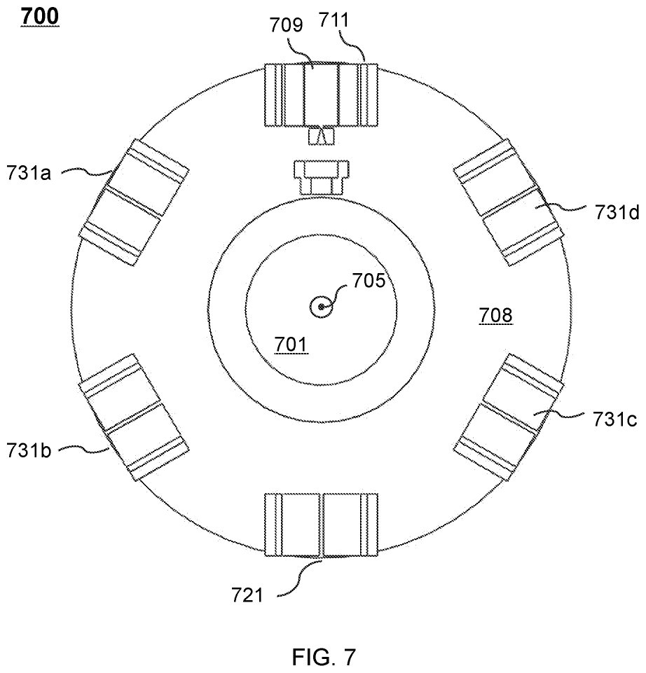

The accelerating waveguide (tube) of t the curved beam deflection unit 509 may be at least partially surrounded by the shielding structure 511. In some embodiments, the shielding structure 511 may provide a cavity coaxial with the longitudinal axis of the tube of the curved beam deflection unit 509, with at least one end being open to let through the radiation beam emitted from the curved beam deflection unit 509. In some embodiments, the shielding structure 511 may have any configuration. For example, the shielding structure 511 may include one annulus on the left side of the recess (i.e., the side near the left chamber) and one annulus on the right side of the recess (i.e., the side near the right chamber) with plates connecting the two annuluses. Alternatively, the annuluses may be replaced by separate arc segments. It should be noted that the shielding structure 511 may be of any shapes provided that at least one end of the shielding structure 511 is open for the radiation beam emitted from the curved beam deflection unit 509 to pass. Details regarding the exemplary configurations of the shielding structure 511 may be found elsewhere in the disclosure (e.g., FIGS. 6-7 and the descriptions thereof).

In some embodiments, the shielding structure 511 may include a plurality of shielding layers. At least one of the plurality of shielding layers may be used to reduce the magnetic interference between one or more components of the MRI device and the radiation therapy device. For example, the shielding structure 511 may include a magnetic shielding layer configured to shield the magnetic field produced by the MRI device (e.g., the main magnetic coils, the shielding magnetic coils, the gradient coils) in case that the electrons may be influenced by the magnetic field.

Additionally, at least one of the plurality of shielding layers may be used to reduce the RF and/or microwave interference between one or more components of the MRI device and the radiation therapy device. For example, the shielding structure 511 may include an electromagnetic shielding layer configured to shield the RF signals produced by the MRI device (e.g., the RF coils) and the microwave produced by the radiation therapy device.

The plurality of shielding layers may be made of a same material and/or different materials. For example, both the electromagnetic shielding layer and the magnetic shielding layer may be made of a material of high magnetic susceptibility and permeability (e.g., non-oriented silicon steel), or one of the electromagnetic shielding layer and the magnetic shielding layer is made of a material of high electric conductivity and magnetic permeability. In some embodiments, the plurality of shielding layers may be magnetically and/or electrically isolated from each other with a suitable dielectric material, such as air or plastic, between them.

Additionally or alternatively, at least one of the plurality of shielding layers may be used to protect one or more components of the MRI device from the radiation produced by the curved beam deflection unit 509. For example, one shielding layer of the plurality of shielding layers may be made of a material that is able to absorb the radiation produced by the radiation beam of the curved beam deflection unit 509. Exemplary materials that are able to absorb the radiation may include a material for absorbing a photon ray and/or a material for absorbing a neutron ray. Exemplary materials for absorbing a photon ray may include steel, aluminum, lead, tungsten, etc., an alloy thereof, or a combination thereof. Exemplary materials for absorbing a neutron ray may include boron, graphite, etc., an alloy thereof, or a combination thereof. It should be noted that, in some embodiments, the shielding structure 511 may be made of only a radiation absorbing material that lacks the property of high magnetic susceptibility and permeability. In this way, the shielding structure 511 may provide only radiation shielding for one or more components of the MRI device.

The target 504 may be configured to receive the accelerated charged subatomic particles or ions (e.g., an electron beam) to produce the radiation beam for the therapeutic radiation. For example, the electron beam may collide with the target 504 to generate high-energy X-rays according to the bremsstrahlung effect. In some embodiments, the target 504 may be located near the exit window of the curved beam deflection unit 509 to receive the accelerated electron beam. In some embodiments, the target 504 may be made of materials including aluminum, copper, silver, tungsten, or the like, or an alloy thereof, or any combination thereof. For instance, the target 504 may be made of a composite material including tungsten and copper, a composite material including tungsten and silver, a composite material including tungsten and aluminum, or the like, or any combination thereof.

The radiation beam from the target 504 may pass through the collimator 512 to form a beam of a specific shape (e.g., cone beam). In some embodiments, the collimator 512 may include a primary collimator, a flattening filter and at least one secondary collimator.

The MLC 510 may be configured to reshape the radiation beam. For example, the MLC 510 may adjust the irradiating shape, the irradiating area, etc., of the radiation beam. The MLC 510 may be placed anywhere on the path of the radiation beam. For example, the MLC 510 may be placed close to the curved beam deflection unit 509 as shown in FIG. 5. Thus, the radiation beam, after being reshaped by the MLC 510, may further pass through the neck portion of the cryostat 503 and the gap 506 between the plurality of main magnetic coils to arrive at the treatment region. As another example, the MLC 510 may be placed by a relatively long distance away from the curved beam deflection unit 509 such that the MLC 510 may be closer to, e.g., the patient to be radiated.

The MLC 510 may be fixed relative to the curved beam deflection unit 509, thus rotating together with the curved beam deflection unit 509 around the axis 505. The MLC 510 may include a plurality of individual leaves of high atomic numbered materials (e.g., tungsten) moving independently in and out of the path of the radiation beam in order to selectively block it. The shape of the radiation beam may vary when the plurality of individual leaves move in and out, forming different apertures that resemble the cross section of the treatment region or lesion including, e.g., a tumor viewed from an axis of the radiation beam (i.e., the vertical dotted line 516 shown in FIG. 5). In some embodiments, the MLC 510 may include one or more layers of leaves. For example, the MLC 510 may have only one layer of leaves and the height of the MLC 510 along the axis of the radiation beam may be between 7 and 10 centimeters. As another example, the MLC 510 may include two layers and the height of the MLC 510 may be at least 15 centimeters.

FIG. 6 shows a perspective view of an exemplary therapy system 600 according to some embodiments of the present disclosure.

As shown in FIG. 6, the therapy system 600 may include a bore 601, an annular cryostat 603 with an axis 605, a recess 608, a curved beam deflection unit 609, and a magnetic shielding arrangement.

The magnetic shielding arrangement may include a first shielding structure 611 and at least one second shielding structure which include a second shielding structure 631a, a second shielding structure 631b, etc.

In some embodiments, all the shielding structures may be identical to each other. For example, the second shielding structure 631a and the second shielding structure 631b may be made of identical materials and have an identical structure as the first shielding structure 611.

The curved beam deflection unit 609 and the electron gun (not shown) may be surrounded or substantially surrounded by the first shielding structure 611. Specifically, the first shielding structure 611 may include a first plate located on one side of the curved beam deflection unit 609 along the circumferential direction of the recess 608 and a second plate located on the opposite side of the curved beam deflection unit 609 along the circumferential direction of the recess 608. The first plate and the second plate may be symmetrical to each other with respect to the axis of the curved beam deflection unit 609. The first plate and the second plate may form an enclosing structure to surround and/or hold the curved beam deflection unit 609. Each of the two plates may have a shape similar to the symbol “ [symbol] ”, which provides a continuous pathway along the axial direction (i.e., the direction of the axis 605) of the cryostat 603 for the magnetic field to pass through. In that the two plates of the first shielding structure 611 are made of at least one high magnetic susceptibility and/or permeability material, the magnetic field may be conducted by the two plates and kept away from the region formed between them, thus achieving the magnetic shielding for the curved beam deflection unit 609. In some embodiments, each of the two plates may be radially arranged about the axis 605, and at least one side of each of the two plates may point to the axis 605.

In some embodiments, the first plate and the second plate may be connected to each other on both sides of the curved beam deflection unit 609 along the axial direction of the cryostat 603, thus forming a closed loop around the curved beam deflection unit 609. In some embodiments, the first plate and the second plate may be separate from each other on both sides of the curved beam deflection unit 609 along the axial direction of the cryostat 603, thus forming a semi-closed loop substantially around the curved beam deflection unit 609. It shall be noted that the configuration of the first shielding structure 611 is not limited, any other configurations (e.g., a hollow cylinder or other shape with curved sides) may also be used to achieve the magnetic shielding.

In some embodiments, the presence of the first shielding structure 611 within the magnetic field of the MRI device may exert an influence on the magnetic field (e.g., deforming the distribution and/or causing the inhomogeneity of the magnetic field). In order to correct the deformation of the magnetic field caused by the first shielding structure 611, similar shielding structures of the magnetic shielding arrangement, including the second shielding structure 631a, the second shielding structure 631b, etc., may also be placed within the recess 608. In some embodiments, all or at least some of the shielding structures may be identical to each other. For example, the second shielding structure 631a and the second shielding structure 631b may be made of an identical material and have an identical structure as the first shielding structure 611. The first shielding structure 611, the second shielding structure 631a, the second shielding structure 631b, etc., may be mounted on the gantry or the drum (not shown) of therapy system 600 to achieve the synchronous rotation with the curved beam deflection unit 609.

In some embodiments, the first shielding structure 611, the second shielding structure 631a, the second shielding structure 631b, etc., may be placed at selected symmetrical circumferential locations about the axis 605 within the annular recess 608. For example, all the shielding structures may be evenly distributed within the recess 608. Each shielding structure may correspond to an opposite or opposing counterpart. Each shielding structure and its counterpart may be symmetrical about the axis 605. As used herein, two shielding structures may be regarded as opposite or opposing if the two shielding structures are symmetric about the axis 605. As used herein, two of shielding structures may be regarded as opposite or opposing if the two shielding structures are symmetric about the axis 605.

FIG. 7 shows the cross-sectional view of the therapy system 700 viewed along the axial direction (i.e., the Z direction) of the cryostat according to some embodiments of the present disclosure.

As shown in FIG. 7, the first shielding structure 711, the second shielding structure 721, and the second shielding structures 731a, 731b, 731c, 731d may be evenly distributed within the recess 708 and around the axis 705 of the bore 701. The distances between each two adjacent shielding structures may be the same. The second shielding structure 721 may be the opposite or opposing counterpart of the first shielding structure 711. The second shielding structure 721 may be identical to the first shielding structure 711 if it rotates clockwise 180 degrees to the position of the first shielding structure 711 around the axis 705. All the shielding structures may be fixed with respect to the curved beam deflection unit 709, and thus may rotate synchronously with the curved beam deflection unit 709.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by the present disclosure, and are within the spirit and scope of the exemplary embodiments of the present disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, for example, an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

We claim:

1. A radiation therapy device, comprising:
an electron gun and a beam deflection unit, wherein
the beam deflection unit is curved and configured to accelerate an electron beam emitted from the electron gun within a magnetic field.

2. The radiation therapy device of claim 1, wherein different portions of the beam deflection unit have different curvatures.

3. The radiation therapy device of claim 2, wherein a curvature of a first end of the beam deflection unit close to the electron gun is greater than a curvature of a second end of the beam deflection unit away from the electron gun.

4. The radiation therapy device of claim 1, wherein:
the beam deflection unit includes a plurality of acceleration cavities arranged in series, and curvatures of the plurality of acceleration cavities from close to the electron gun outward decrease sequentially.

5. The radiation therapy device of claim 4, wherein a deflection angle of an electron beam traversing one of the plurality of acceleration cavities is from 0° to 15°.

6. The radiation therapy device of claim 4, wherein the plurality of acceleration cavities include a first acceleration cavity, a second acceleration cavity, a third acceleration cavity, and a fourth acceleration cavity arranged in series from close to the electron gun outward.

7. The radiation therapy device of claim 6, wherein:
a first deflection angle of an electron beam traversing the first acceleration cavity is from 0° to 10°,
a second deflection angle of the electron beam traversing the second acceleration cavity is from 0° to 15°,
a third deflection angle of the electron beam traversing the third acceleration cavity is from 0° to 5°, and
a fourth deflection angle of the electron beam traversing the fourth acceleration cavity is from 0° to 5°.

8. The radiation therapy device of claim 1, wherein a length of the beam deflection unit is in a length range of 200 mm to 400 mm.

9. The radiation therapy device of claim 1, wherein a deflection angle of an electron beam traversing the beam deflection unit is from 0° to 30°.

10. The radiation therapy device of claim 1, wherein an intensity of the magnetic field is from 0 Gs to 50 Gs.

11. The radiation therapy device of claim 1, wherein the electron gun includes a radio frequency electron gun.

12. The radiation therapy device of claim 11, wherein the radio frequency electron gun includes a hot cathode disposed in the beam deflection unit.

13. The radiation therapy device of claim 12, wherein the hot cathode is disposed at an end of the beam deflection unit close to the electron gun.

14. A magnetic resonance guided radiation therapy system, the system comprising a radiation therapy device and a magnetic resonance imaging (MRI) device, wherein:
the radiation therapy device includes an electron gun and a beam deflection unit, and the beam deflection unit being curved and configured to accelerate an electron beam emitted from the electron gun within a magnetic field, and
the MRI device includes:
a main magnet body including a plurality of main magnetic field coils coaxially arranged along an axis; and
a plurality of shielding coils including a first shielding coil, a second shielding coil and a shielding coil group arranged coaxially along the axis, wherein the shielding coil group is located between the first shielding coil and the second shielding coil.

15. The system of claim 14, wherein the shielding coil group includes a first coil group and a second coil group arranged coaxially along the axis; and
the first coil group or the second coil group includes a first coil and a second coil.

16. The system of claim 15, wherein a direction of a current within the first coil is opposite to a direction of a current within the second coil; and/or
a radius of the first coil or the second coil is larger than that of the plurality of main magnetic field coils; and/or
a radius of the first coil is greater than a radius of the second coil.

17. A magnetic resonance guided radiation therapy system, the system comprising a radiation therapy device and a magnetic resonance imaging (MRI) device, wherein:
the MRI device includes:
a plurality of main magnetic coils;
a plurality of shielding magnetic coils; and
an annular cryostat in which the plurality of main magnetic coils and the plurality of shielding magnetic coils are coaxially arranged along anaxis of the annular cryostat, the plurality of shielding magnetic coils being arranged at a larger radius from the axis than the plurality of main magnetic coils, the annular cryostat including at least one outer wall and at least one inner wall coaxial around the axis, the annular cryostat including an annular recess between the at least one outer wall and the at least one inner wall, and the annular recess having an opening formed at the at least one outer wall;
the radiation therapy device includes an electron gun and a curved beam deflection unit, and the beam deflection unit is configured to accelerate an electron beam emitted from the electron gun within a magnetic field, the beam deflection unit being at least partially located within the annular recess of the annular cryostat;
a first shielding structure configured to provide magnetic shielding for the electron gun and the beam deflection unit; and
at least one second shielding structure substantially identical to the first shielding structure, wherein the first shielding structure and the at least one second shielding structure are respectively located at selected circumferential locations within the annular recess.

18. The system of claim 17, wherein the at least one second shielding structure is located at an opposite circumferential position of the first shielding structure with respect to the axis.

19. The system of claim 17, wherein the electron gun and the curved beam deflection unit is at least partially surrounded by the first shielding structure.

20. The system of claim 17, wherein the at least one second shielding structure includes more than two second shielding structures, and the first shielding structure and the at least one second shielding structure are evenly distributed within the annular recess.
